(19) United States
(12) Patent Application Publication
(10) Pub. No.: US 2005/0148807 A1
(43) Pub. Date: Jul. 7, 2005

(54) Magneto-massage system

(75) Inventors: Igal Salkinder, Petah Tikva (IL); Bernardo Miller, Haifa (IL)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: PHYMAG LTD., Hefer (IL)

(21) Appl. No.: 10/505,283

(22) Filed: Aug. 18, 2004

Related U.S. Application Data
(63) Continuation of application No. PCT/IL02/00543, filed on Jul. 3, 2002.

(30) Foreign Application Priority Data
Jul. 13, 2001 (IL) 144317

(51) Int. Cl. A61N 2/00; A61B 17/52; A61H 1/00; A61H 1/02; A61H 5/00

(52) U.S. Cl. 600/9; 601/15

(57) ABSTRACT

A treatment system for providing electromagnetic therapy and massage. The treatment system includes an electromagnetic field inductor for producing an electromagnetic field, and at least one magnetic ball. The magnetic ball includes a central magnet core covered in a nonmagnetic material, wherein the electromagnetic field is capable of causing the magnetic ball to move freely and randomly within the electromagnetic field so as to massage a body part placed within the electromagnetic field.

This application is a Continuation of PCT/IL02/00543 filed Jul. 3, 2002, the entire contents of which are incorporated herein by reference.

### FIELD AND BACKGROUND OF THE INVENTION

The present invention relates to a physiotherapeutic system and, more particularly, to a system that uses a pulsating magnetic field for induced massage combined with pulse magnetic field therapy. Such a system finds use in the treatment of individuals with a range of clinical problems including vascular, orthopedic, rheumatologic, neurologic, and dermatologic disorders. Diabetes mellitus is a chronic disorder that is characterized by hyperglycemia, and associated with major abnormalities in carbohydrate, fat, and protein metabolism. It is accompanies by a marked tendency to develop renal, ocular, neurologic and premature cardiovascular disorders. The two major types of diabetes are Type I or insulin dependent diabetes mellitus (IDDM) and Type II or non-insulin dependent diabetes mellitus (NIDDM).

More than 125 million people are affected by diabetes worldwide. Between 2% and 15% of Western population have diabetes. The modern “western” lifestyle is precipitating a global epidemic of diabetes. The number of diabetics is increasing by approximately 4%-5% per year and it is estimated that as many as 40% of people aged 65 and over have either type II diabetes or its precursor state glucose intolerance. The number of diabetes patients is expected to double over the next 10 to 15 years. Diabetes costs the American economy, for instance, over US $100 billion annually. Healthcare expenditures in the United States for people with diabetes represent approximately 15% of the total annual budgets of health maintenance organizations. (Medical Healthcare Marketplace Guide, 16‘x’ ed., 2000-2001)

One of the major complications of diabetes is peripheral vascular disease, particularly of the lower extremities. Lower extremity arterial disease (LEAD) is identified clinically by intermittent claudication and/or absence of peripheral pulses in the lower legs and feet, representing decreased arterial perfusion of the extremity. In population-based studies, pulse deficits were found in −10% of diabetic subjects, absent foot pulses in −20% −30%, and intermittent claudication in −9%.

Lower extremity ulcers and amputations are an increasing problem among individuals with diabetes. The annual incidence of foot ulcers in community-based studies was 2%-3%, and prevalence was 4% 10%. 0%. In the 1983-90 National Hospital Discharge Surveys (NHDS), 6% of hospital discharge records that listed diabetes also listed a lower extremity ulcer condition, and chronic ulcers were present on 2.7%. The average length of stay for diabetes discharges with ulcer conditions was 59% longer than for diabetes discharges without them. Clinical epidemiological studies suggest that foot ulcers precede ˜85% of nontraumatic lower extremity amputations in individuals with diabetes.

The incidence of amputation in patients with diabetes is ˜0.4%-0.8% per year. About half of amputations in the United States occur in people with diabetes. NHDS data indicate there were an annual average of ˜54,000 hospital discharges listing diabetes and a nontraumatic lower extremity amputation in 1989-92. Lower level amputations (toe, foot, and ankle) comprised 55%. Hospital discharge data indicate that 9%-20% of amputees experienced a second amputation within 12 months. By 5 years after an initial amputation, 28%-51% had undergone a second amputation. Perioperative mortality among diabetic amputees averaged 5.8% in the 1989-92 NHDS. Five-year mortality following amputation was 39%-68% in other studies.

It would be desirable to have a system that would result in the postponement of amputation of the lower extremities in cases of diabetes mellitus, by improving the circulatory status of affected individuals.

The beneficial therapeutic effects of massage and of pulsating magnetic fields individually have been well known to medicine for many years; however, previous attempts to combine them into a single therapeutic apparatus have been limited. Pulsed Electromagnetic Field Therapy (PEMF) is a non-invasive treatment that works by permeating the body with a pulsed magnetic field, reaching every cell and changing cell potentials, and influencing the activity of enzymes and coenzymes by stimulating paramagnetic ions. The application of an external electromagnetic field causes the ions (charged atoms) always present in the body to travel outward toward the sides of the blood vessel walls. This increased blood flow brings more oxygen and nutrients to the cells, removing metabolic by-products from the tissues and promoting alkaline reactions (i.e., pH balance). In an ailing limb the application of the electromagnetic field speeds up recovery by improving blood flow. Therapy with an electromagnetic field has been found to be particularly effective in dermatology (for treatment of such clinical problems as decubitus ulcers, skin necrosis, burns, cicatrization defects, and the like), rheumatology, pain therapy, physiatrics, sports medicine, rehabilitation and orthopedics. Since there are many documented scientific results to prove its efficiency in various conditions this therapy method is considered complementary medicine. It is to be distinguished from magnet therapy (also known as biomagnetic therapy) which usually refers to a constant magnitude (and vector) magnetic field, created by permanent magnets (natural and artificial). The practitioners of this technology usually have a preference for one magnetic pole (the north) regarding its expected influence on the human body. This therapy is considered alternative medicine.

The following discussion of PEMF is based on an analysis of the scientific basis of PEMF by Ramey (Ramey, D W (1998), Analysis—Magnetic and Electromagnetic Therapy, in The Scientific Review of Alternative Medicine, Prometheus Books).

Extracellular matrix synthesis and repair are subject to regulation both by chemical agents (such as cytokines and growth factors) and physical agents, principally mechanical and electrical stimuli. The precise nature of such electromechanical signals is not known, however. In bone, mechanical and electrical signals may regulate the synthesis of extracellular matrix by stimulating signaling pathways at the cell membrane. (Davidovitch, Z., et al. Biochemical mediators of the effects of mechanical forces in electric currents on mineralized tissue. Calcif Tissue Int 36: s86-s79, 1984; and Aaron, R. and Ciombor, D. Acceleration of Experimental Endochondral Ossification by Biophysical Stimulation of the Progenitor Cell Pool. J Orthop Res 14(4): 582-89, 1996.) In soft tissue, alternating current electrical fields induce a redistribution of integral cell membrane proteins which, hypothetically, could initiate signal transduction cascades and cause a reorganization of cytoskeletal structures (Cho, M., et al. Reorganization of microfilament structure induced by ac electric fields. FASEB J 10: 1552-1558, 1996.)

There is ample evidence that electrical activity exists in the body at all times. For example, electrical currents can be measured in the beating heart and are also generated in the production of bone. Endogenous electrical current densities produced by mechanical loading of bone under physiologic conditions approximate 1 Hz and 0.1-1.0 microA/cm² (MacGinitie, L. A., Gluzbank, Y. A. and Grodzinski, A. J. Electric Field Stimulation can Increase Protein Synthesis in Articular Cartilage Explants. J Orthop Res 12: 151-60, 1994). Thus, it is theorized that application of an appropriate electrical current, either directly through wires or indirectly through induction by a magnetic field, may affect tissues in several ways. Cells and tissues respond to a variety of electrical signal configurations in ways that suggest a degree of specificity for both the tissue affected and the signal itself.

The most widely studied application of electromagnetic field therapy in human medicine is in fracture therapy. Although the mechanisms remain undetermined, several studies report that electrical fields generated by pulsating electromagnetic field therapy stimulate biologic processes pertinent to osteogenesis (Shimizu, T., et al. Bone ingrowth into porous calcium phosphate ceramics; influence of pulsating electromagnetic field. J Orthop Res 6: 248-258, 1988; Rubin, C, McLeod, K and Lanyon, L. Prevention of osteoporosis by pulsed electromagnetic fields. J Bone Joint Surg [Am] 71: 411-416, 1989; and Cruess, R. and Bassett, C A L. The effect of pulsing electromagnetic fields on bone metabolism in experimental disuse osteoporosis. Clin Orthop 173: 345-250, 1983. 10,11,12 and bone graft incorporation). This form of therapy is approved for the treatment of delayed and non-union fractures in humans in the U.S. by the United States Food and Drug Administration. Effectiveness of the treatment is supported by at least two double-blind studies (Sharrard, W. A double blind trial of pulsed electromagnetic fields for delayed union of tibial fractures. J Bone and Joint Surg [Br] 72: 347-355, 1990; and Mooney, V. A randomized double blind prospective study of the efficacy of pulsed electromagnetic fields for interbody lumbar fusions. Spine 15: 708-712, 1990).

Pulsating electromagnetic field therapy has also been evaluated in the treatment of soft tissue injuries, with the results of some studies providing evidence that this form of therapy may be of value in promoting healing of chronic wounds, such as decubitus ulcers (leran, M., et al. Effect of Low Frequency Pulsing Electromagnetic Fields on Skin Ulcers of Venous Origin in Humans: A Double-Blind Study. J Orthop Res 8(2): 276-282, 1990), in neuronal regeneration ( Kort, J., Ito, H. and Basset, C. A. L. Effects of pulsing electromagnetic fields on peripheral nerve regeneration. J Bone Jt Sug Orthop Trans 4: 238, 1980; and Sisken, B. F., et al. Pulsed electromagnetic fields stimulate nerve regeneration in vitro and in vivo. Restorative Neurology and Neuroscience 1: 303-309, 1990b), and in many other soft tissue injuries (Polk, C. Electric and Magnetic Fields for Bone and Soft Tissue Repair. In, Handbook of Biological Effects of Electromagnetic Fields, 2nd ed. Polk, C. and Postow, E., eds. CRC Press, Boca Raton, Fla., 231-246, 1996; and Bassett, C. A. L. Beneficial Effects of Electromagnetic Fields. J of Cell Biochem 51: 387-393, 1993).

The direct mechanical effect of rhythmically applied pressure by massage also increases the blood flow rate. It stimulates the nerve receptors and causes the blood vessels to dilate, allowing greater fluid exchange (i.e., blood, oxygen and lymph, and the removal of waste products such as lactic acid and carbon dioxide). Massage technology is well established with universal acceptance of its efficacy. Massage has proven beneficial in many fields, including orthopedics, traumatology, rheumatology, treatment of complicated and simple fractures, treatment of wounds, burns and degenerative diseases, coronary and circulation diseases and disorders of the neurological system. Massage therapy may be applied in any of several ways ranging from manual treatment by a massage therapist to use of a mechanical device to assist in the application of pressure and motion.

It would be desirable to have a therapeutic system that combines the beneficial effects of magnetic field therapy and massage for the treatment of circulatory disorders such as diabetes induced peripheral arterial disease and its complications as well as for other medical treatments, for use with humans and non-human animals.

A number of devices have been previously described for generating an electromagnetic field for providing electromagnetic therapy, and are disclosed, for example, in U.S. Pat. No. 4,066,065 issued to Kraus; U.S. Pat. No. 4,765,310 issued to Deagle et al.; U.S. Pat. No. 4,838,850 issued to Rosengart; U.S. Pat. No. 5,088,976 issued to Liboff et al.; U.S. Pat. No. 5,267,939 issued to Liboff, et al.; and U.S. Pat. No. 5,880,661 issued to Davidson, et al. Similarly, a number of patents disclose mechanical devices for assisting in providing massage therapy, including, for example, U.S. Pat. No. 3,585,990 issued to Blachly et al.; U.S. Pat. No. 3,636,945 issued to Sato; U.S. Pat. No. 5,123,406 issued to Masuda; and U.S. Pat. No. 5,462,515 issued to Tseng.

U.S. Pat. No. 6,102,875 issued to Jones teaches a handheld apparatus for simultaneously applying massage and biomagnetic therapy. This device uses a permanent magnet of fixed (rather than alternating) polarity. Massage balls are attached to the head of the device and are rotated by the action of an electric motor. An electromechanical or electromagnetic vibrator can impart vibrational motion to the balls. Such a device suffers from a number of limitations. It is handheld and imparts a local effect only on the limited portion of the body with which it is placed in contact, it imparts only kneading and vibrational massage, and it utilizes fixed biomagnetic therapy rather than a pulsed electromagnetic field.

Another combination device is that taught in U.S. Pat. No. 5,084,003 issued to Susic. That device includes a coil that generates an electromagnetic field and small permanent magnetic plates arranged inside a cover, apron or bandage and flexibly connected to each other placed within the coil. The plates undulate or vibrate to provide a massage effect. Such a device suffers from several limitations. The magnetic plates are fixed in a particular arrangement and thus only limited, non-random and uniform movement of the plates is possible, imparting only a vibrational, flat type of massage. Because the plates are relatively fixed in position, the polarity of these plates must be fixed (for example along the longitudinal axis of the plates, or in parallel orientation to one another), and is neither variable nor randomly variable. It would be advantageous to have a system in which the elements which impart the massaging action are not fixed in a particular arrangement allowing freedom of movement so as to impart massaging action other than flat vibrational, undulating or kneading motion, such as percussion. The use of percussion produces a deeper massage than vibration and is therefore better able to improve the circulation. Percussion massage also stimulates the neural fibers in a randomized pumping action that results in the reduction of pain. Percussion massage results in a skin sensitive reaction that promotes peripheral vasodilatation and increases the subepithelial circulation. The skin effect promoted initially by vibrational massage loses its effectiveness rapidly, in a very short time, due to an accommodation effect of the sensitive nervous system.

The device taught in U.S. Pat. No. 5,084,003 issued to Susic is large, includes a bed, and is not readily mobile but rather stationary. Further no control element is provided which can allow monitoring of the treatment, safety monitoring and regulation, automated function with variation of treatment parameters, programmable (locally or remotely) functioning, or, selection from (and changing of) a memory of predefined treatment programs.

There is thus a widely recognized need for, and it would be highly advantageous to have, a therapeutic system that combines massage and pulse magnetic field therapy devoid of the above limitations.

### SUMMARY OF THE INVENTION

According to the present invention there is provided a treatment system for providing electromagnetic therapy and massage including, (a) an electromagnetic field inductor for producing an electromagnetic field, and (b) at least one magnetic ball, the magnetic ball having a central magnet core covered in a substantially nonmagnetic material, wherein the electromagnetic field is capable of causing the at least one magnetic ball to move freely and randomly within the electromagnetic field so as to massage a body part placed within the electromagnetic field.

According to further features in preferred embodiments of the invention described below, the electromagnetic field has a maximal induction of 200 Gauss.

According to further features in preferred embodiments of the invention described below, the electromagnetic field is pulsed.

According to still further features in the described preferred embodiments, the electromagnetic field is intermittent.

According to yet further features in the described preferred embodiments, the system the electromagnetic field is alternating in polarity.

According to additional features in the described preferred embodiments below, the electromagnetic field alternates in polarity with a frequency of 50 to 60 Hertz.

According to still further features in preferred embodiments of the invention as described below, the electromagnetic field alternates in polarity with sinusoidal oscillations.

According to still further features in preferred embodiments of the invention as described below, the body part is of an animal.

According to still further features in preferred embodiments of the invention as described below, the body part is of a human.

According to still further features in preferred embodiments of the invention as described below, the body part is an entire body.

According to still further features in preferred embodiments of the invention as described below, the body part is a limb.

According to still further features in preferred embodiments of the invention as described below, the at least one magnetic ball is a plurality of magnetic balls.

According to still further features in preferred embodiments of the invention as described below, the at least one magnetic ball is spherical.

According to still further features in preferred embodiments of the invention as described below, the shape of the at least one magnetic ball is selected from the group consisting of cube, cylinder, cone, pyramid, rectangular prism, and irregular polyhedral solid.

According to still further features in preferred embodiments of the invention as described below, the at least one magnetic ball has at least one projection extending from a surface of the at least one magnetic ball.

According to still further features in preferred embodiments of the invention as described below, the substantially nonmagnetic material is soft.

According to still further features in preferred embodiments of the invention as described below, the substantially nonmagnetic material is hard.

According to still further features in preferred embodiments of the invention as described below, the substantially nonmagnetic material is selected from the group consisting of plastic, rubber, silicone epoxy, foam rubber and fabric.

According to still further features in preferred embodiments of the invention as described below, the system further includes a housing, the housing including at least one wall and a base surface, the at least one wall and the base surface enclosing a bath, wherein the electromagnetic field inductor surrounds the bath, and the at least one magnetic ball moves freely and randomly within the bath.

According to still further features in preferred embodiments of the invention as described below, the system further includes a cover above the bath, the cover having a passage therein for insertion of the body part.

According to still further features in preferred embodiments of the invention as described below, the cover is transparent.

According to still further features in preferred embodiments of the invention as described below, the electromagnetic field inductor is circular.

According to still further features in preferred embodiments of the invention as described below, the electromagnetic field inductor is located within a housing mounted on a stand, the housing having a central aperture adapted to enable insertion of the body part.

According to still further features in preferred embodiments of the invention as described below, the position of the electromagnetic field inductor on the stand may be adjusted.

According to still further features in preferred embodiments of the invention as described below, the housing has a lining rest.

According to still further features in preferred embodiments of the invention as described below, the at least one magnetic ball is contained within a sleeve, the sleeve being adapted so as to be placeable around the body part.

According to still further features in preferred embodiments of the invention as described below, the sleeve has a plurality of chambers, each of the plurality of chambers having an enclosure sack with at least two portions, the first portion in contact with the body part, and a second part attached to the first part.

According to still further features in preferred embodiments of the invention as described below, the first portion of the enclosure sack is soft, and the second portion is firmer than the first portion.

According to still further features in preferred embodiments of the invention as described below, the sleeve has at least one vent for changing the at least one magnetic ball therein.

According to still further features in preferred embodiments of the invention as described below, the sleeve is transparent.

According to still further features in preferred embodiments of the invention as described below, the system further includes a control element for automated operation of the system.

According to still further features in preferred embodiments of the invention as described below, the control element is programmable, such that at least one parameter of the operation of the system may be changed by an operator of the system.

According to still further features in preferred embodiments of the invention as described below, the at least one parameter is selected from the group consisting of maximal intensity of the electromagnetic field, pulse cycle time, pause time, and total treatment duration.

According to still further features in preferred embodiments of the invention as described below, the at least one parameter may be changed using at least one remote program input device connected to the control element by at least one communication channel.

According to still further features in preferred embodiments of the invention as described below, the at least one communication channel is selected from the group consisting of a telephone connection, a cellular telephone connection, an infrared connection, a satellite connection, cables connection, an Internet connection, a local area network connection and a radio frequency connection.

According to still further features in preferred embodiments of the invention as described below, the control element is adapted to perform an emergency stop.

According to still further features in preferred embodiments of the invention as described below, the system further includes a remote input device for causing the control element to perform the emergency stop.

According to still further features in preferred embodiments of the invention as described below, the remote input device conveys input to the control element by a means selected from the group consisting of electrical impulses traveling along a wire, wireless transmission, sonic transmission, infrared transmission, ultrasound transmission, microwave transmission and radio frequency transmission.

According to still further features in preferred embodiments of the invention as described below, the control element is adapted so as to prevent operation of the system if at least one safety parameter is exceeded.

According to still further features in preferred embodiments of the invention as described below, the at least one safety parameter is selected from the group consisting of inductor temperature and current intensity.

According to still further features in preferred embodiments of the invention as described below, the system is used for the treatment of vascular disease.

According to still further features in preferred embodiments of the invention as described below, the system is used for the treatment of inflammatory conditions.

According to still further features in preferred embodiments of the invention as described below, the system is used for the treatment of pain.

The present invention successfully addresses the shortcomings of the presently known configurations by providing a system that uses a pulsed electromagnetic field for induced massage with random percussion combined with pulse magnetic field therapy.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic diagram illustrating the principles and activity of the present invention;

FIG. 2 is a simplified block diagram showing the general components of a preferred embodiment of the system according to the present invention;

FIG. 3 is a perspective view of a preferred embodiment of the system of the present invention;

FIG. 4 is a schematic longitudinal cross-section illustrating a preferred embodiment of the system of the present invention;

FIG. 5 is a schematic longitudinal cross-section illustrating an alternate preferred embodiment of the system of the present invention;

FIG. 6 is a perspective view of an alternate preferred embodiment of the system of the present invention;

FIG. 7 is a perspective diagram showing an alternate preferred embodiment of the system of the present invention;

FIG. 8 illustrates an alternate preferred embodiment of the system of the present invention;

FIG. 9 is a coronal cross section of an alternate preferred embodiment of the system of the present invention, including a sleeve;

FIG. 10 is a perspective diagram showing an alternate preferred embodiment of the system of the present invention, including a sleeve;

FIG. 11 is a diagram of an alternate preferred embodiment of a sleeve of the system according to the present invention;

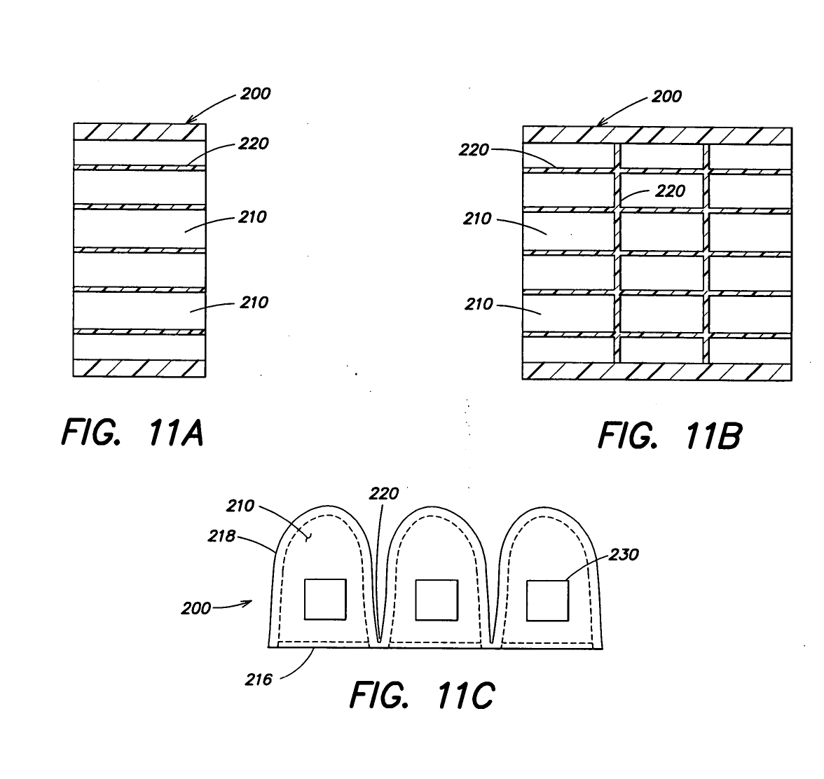

FIG. 12 is a block diagram of the electronics of a preferred embodiment of the present invention; and,

FIG. 13 is a schematic diagram of a preferred embodiment of a ball according to the present invention.

### DESCRIPTION OF THE PREFERRED EMBODIMENTS

The present invention is of a system that uses a pulsed electromagnetic field for induced random percussion massage combined with pulsed magnetic field therapy that can be used for physiotherapeutic treatment. Specifically, the present invention can be used to increase circulatory flow in the treatment of vascular disease, such as diabetes mellitus induced peripheral vascular disease.

The principles and operation of a magneto-massage system according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

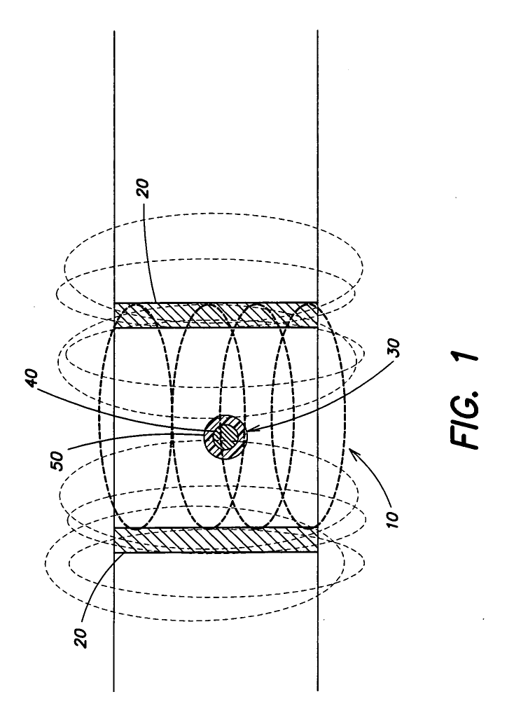

Referring now to the drawings, FIG. 1 illustrates the principles underlying the system of the present invention. A relatively strong pulsed electromagnetic field (10) is created by an electromagnetic induction coil (20). Within field 10 is placed at least one, and preferably a plurality of, massage balls (30) [one is illustrated in FIG. 1] each of which has a central core (40) composed of a magnet, covered with a cushioning material (50). Electromagnetic field 10 is pulsed, and preferably, though not necessarily, alternating. Electromagnetic field 10 thus has a rapidly changing polarity, producing shifting vectors of changing magnitude, and in some cases direction, to apply attraction and repulsion to magnet 40. The pulsing magnetic field thus causes balls 30 within (whose magnetic polarity is randomly oriented) to move freely and randomly and to change movement direction and speed very frequently. Electromagnetic field 10 is also intermittent in certain preferred embodiments, and therefore sometimes is present (“on”) and sometimes is absent (“off”), at changeable, controllable duty cycles. During the “off” periods, the effect of other forces (such as gravity) acting on balls 30 becomes greater, further contributing to the randomness of position and orientation of central magnet core 40 and ball 30. Massage balls 30 impact on and bounce off coil 20 and any other object placed within field 10. A human or animal body or body part, such as a limb, may be placed within field 10. The body part positioned within field 10 will thus receive the therapeutic effects of pulsed electromagnetic field 10 and as well will have applied a percussion massage through the effect of the physical contact of balls 30 impacting upon the body part.

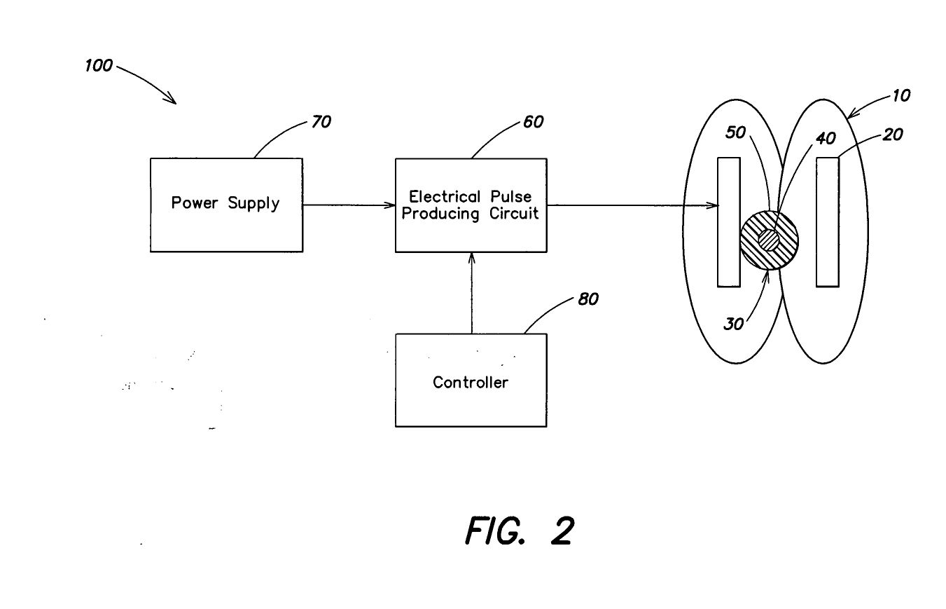

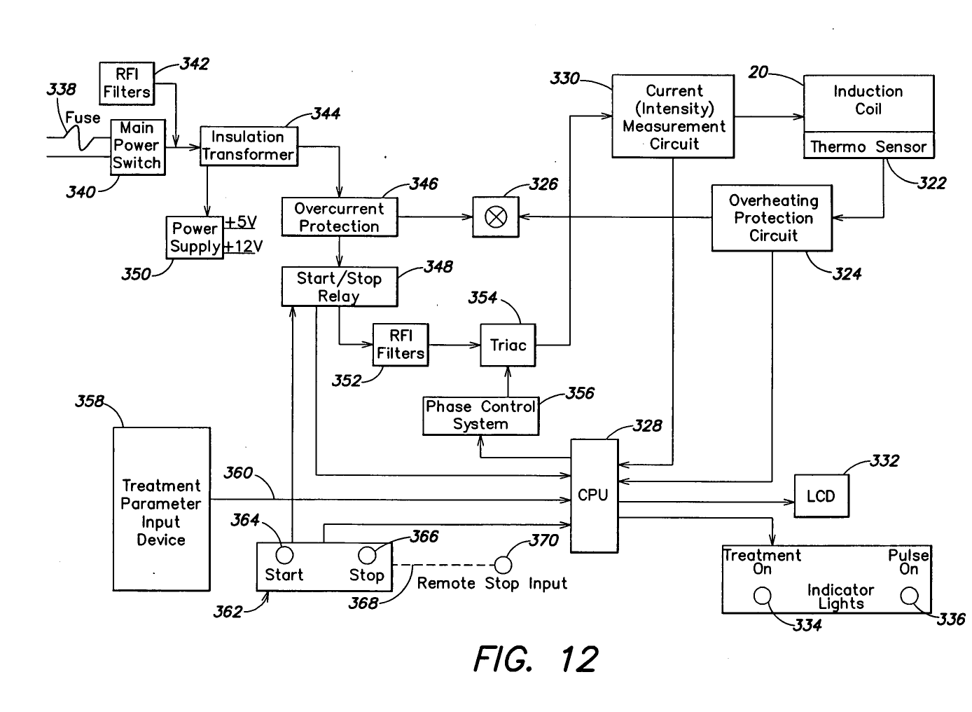

FIG. 2 is a simplified block diagram showing the general components of a preferred embodiment of the magneto-massage system 100 according to the present invention. Electromagnetic field 10 is produced by electromagnetic field induction coil 20 that is connected to pulsed electrical current circuit 60. Circuit 60 produces pulses of alternating phase controlled current such that electromagnetic field 10 produced by coil 20 is pulsed (that is, varying in magnitude of intensity) and preferably, but not necessarily, alternating in polarity. Alternate embodiments in which electromagnetic field 10 produced by coil 20 is pulsed but not alternating in polarity are envisioned as being within the scope of the present invention. The design and operative assembly of circuit 60 and coil 20 are done in accordance with principles commonly known to those of ordinary skill in the art to which this invention pertains. Circuit 60 is further connected to power supply 70 and control element 80 that controls the automated and programmable operation of the entire system 100. In a preferred embodiment of the present invention, described hereinbelow and in FIG. 12, circuit 60 does not utilize a current generator or oscillator, but rather draws the alternating current directly from a 50-60 Hz main power line. Electromagnetic field 10 acts upon at least one, and preferably a plurality of, massage balls (30) placed within field 10 [one is illustrated in FIG. 2], each of which has a central core (40) composed of a magnet, covered with a substantially nonmagnetic cushioning material (50). In certain alternative embodiments, the central core magnet 40 is a permanent magnet, while in others, central core magnet 40 is composed of a magnetically susceptible material (such as iron) that becomes magnetic when placed within an electromagnetic field. Thus central core magnet 40 is at least composed of a magnetic susceptible material such that this material becomes magnetized in an electromagnetic field acting thereon.

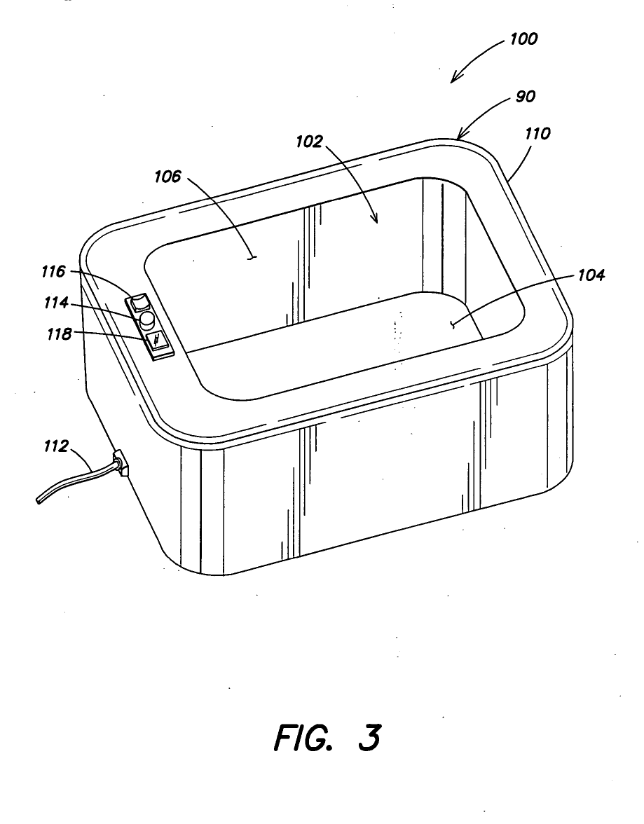

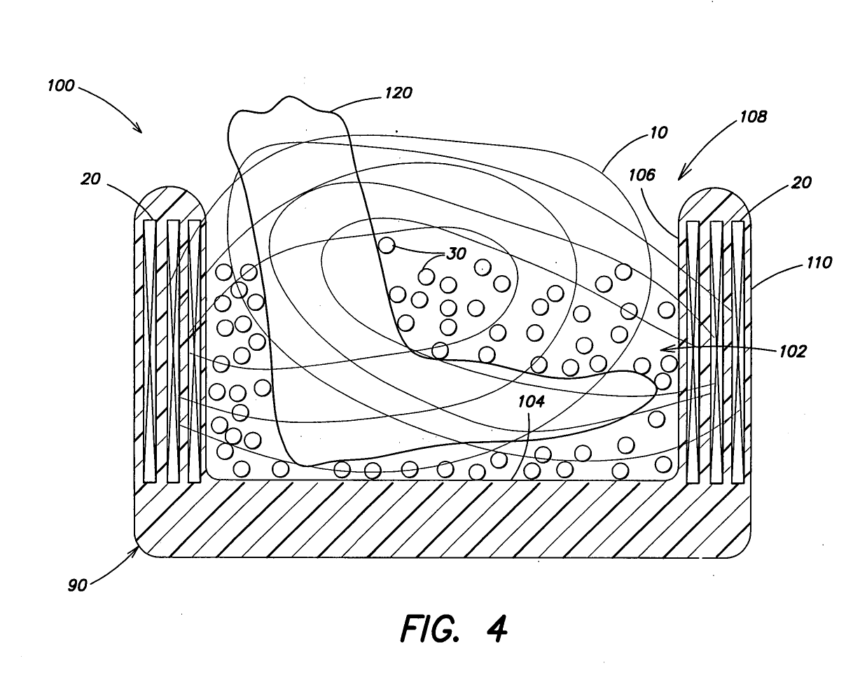

FIG. 3 is a perspective view of a preferred embodiment of the system of the present invention, and FIG. 4 is a schematic longitudinal cross-section illustrating a preferred embodiment of the system of the present invention. Like reference numerals refer to like parts throughout the figures of the drawing The components of system 100 are contained within a housing 90, constructed of a durable material such as a PVC plastic. Housing 90 contains a base surface 104 and inner walls 106 that enclose a hollow interior cavity 102 defining a bath. The upper surface of bath cavity 102 is open, creating a mouth 108. While, for illustrative purposes, housing 90 is shown in FIGS. 3 and 4 as an oblong rectangular prism, housing 90 and bath 102 may be of varying shapes and sizes. Induction coil 20 is located within housing 90, between inner walls 106 and outer walls 110, surrounding bath cavity 102, such that electrical field 10 is produced within bath 102. Power for the system can be provided by connection of system to a main supply of power via power cord 112. An operator, who may be the person being treated, or another individual such as a physical therapist, physician, or nurse, controls operation of system 100 through at least one input device such as button 114 and switch 116. Operation of the system can be monitored using at least one display device, such as light 118.

At least one, and preferably a plurality of, massage balls 30 is placed freely in no particular specified arrangement within bath 102. Balls 30 are agitated into random motion by the effect of the pulsed electromagnetic field 10. A body part 120 to be treated is placed into bath 102, and thus into field 10. Body part 120 may be the entire body or any portion thereof, including particularly, but not limited to, a limb or portion thereof. The body or the part thereof may be that of a human or of an animal. When the system is activated, in addition to the electromagnetic field acting upon the tissues of body part 120, balls 30, moving freely and randomly, will impact upon surfaces 104 and 106 of the system and with body part 120. The physical contact of balls 30, directly or indirectly, with body part 120 will impart a percussion massage on body part 120. The frequency, force and speed of the impact, and the quality of the massage can be changed by altering various parameters, including those of field 10 and of balls 30.

Altering the magnitude of the strength of the field and the frequency of oscillation for example can influence the speed and frequency of the contact. The quality of the massage can be further altered by a change in the number and size of balls 30. In addition other characteristics of balls 30 can be varied in order to alter the quality of the massage.

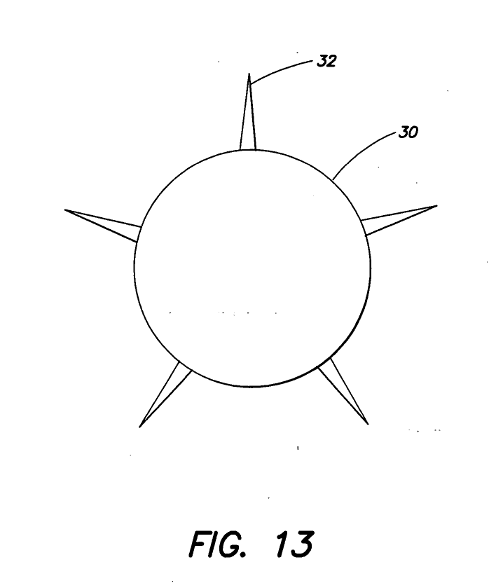

For the purpose of the specification and accompanying claims, use of the term “ball” is meant to encompass projectile objects of shapes other than spheres. It will be appreciated that balls 30 could be of other non-spherical shapes, including, but not limited to, cubes, cylinders, cones, pyramids, rectangular prisms, plates, or irregular polyhedral solids. Further encompassed by the term “balls” are objects whose surface has at least one projection (32) radiating and extending from the surface, including spikes, bumps, and knobs (see FIG. 13). In those embodiments with a plurality of balls 30, the balls are not all necessarily of the same size and shape. The structure of balls 30, regardless of shape is that they consist of a magnetic core 40, made for example of a ferromagnetic material of any varying shape and size covered in some other material, 50. In certain alternative embodiments, the central core magnet 40 is a permanent magnet, while in others, central core magnet 40 is composed of a magnetically susceptible material (such as iron) that becomes magnetic when placed within an electromagnetic field.

Covering material 50 used is variable and in differing configurations is soft or hard, and rigid or flexible. Covering material 50 may be produced from any of a number of, generally synthetic, generally non-conductive materials including plastics (hard and soft), rubber, silicone epoxies, foam rubber, or fabric as non-limiting examples. The size of balls 30 ranges from 1 mm to 10 cm, preferably from 2 mm to 2 cm, and most preferably is between 5 mm and 1 cm. The number of balls 30 can be varied from 1 to a plurality of balls, preferably up to 2000, more preferably 1 to 200, most preferably 5 to 100, depending on the application, device size, configuration and the like. Balls 30 with more irregular shapes with edges and vertices, balls with projections, and coverings that are harder and more rigid can be used for example when more skin reaction is desired. For gentler massage spherical balls of a softer material such as rubber, polystyrene or a polyester may be used. In those embodiments in which central magnet core 40 is a permianent magnet, as each ball 30 comes into proximity with, or contacts, body part 120, the permanent magnet core 40 of the ball 30 exerts a biomagnetic effect on the tissues of body part 120.

In some configurations of the system, the body part 120 to be treated is first placed into a sock of a thin material so that balls 120 do not come into direct physical contact with body part 120 but rather impacts on part 120 through the thin layer of the sock. The thin material may be a plastic or a fabric and is preferably transparent, and serves to prevent contamination of the balls and other parts of the system such as surfaces 104 and 106, so that the components of system 100 do not have to be sterilized between uses.

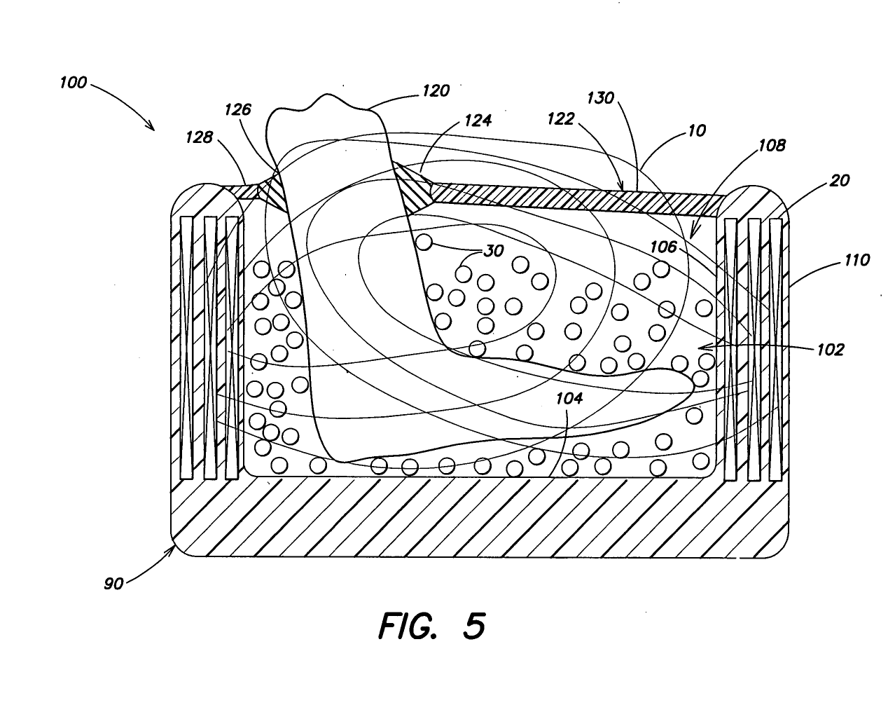

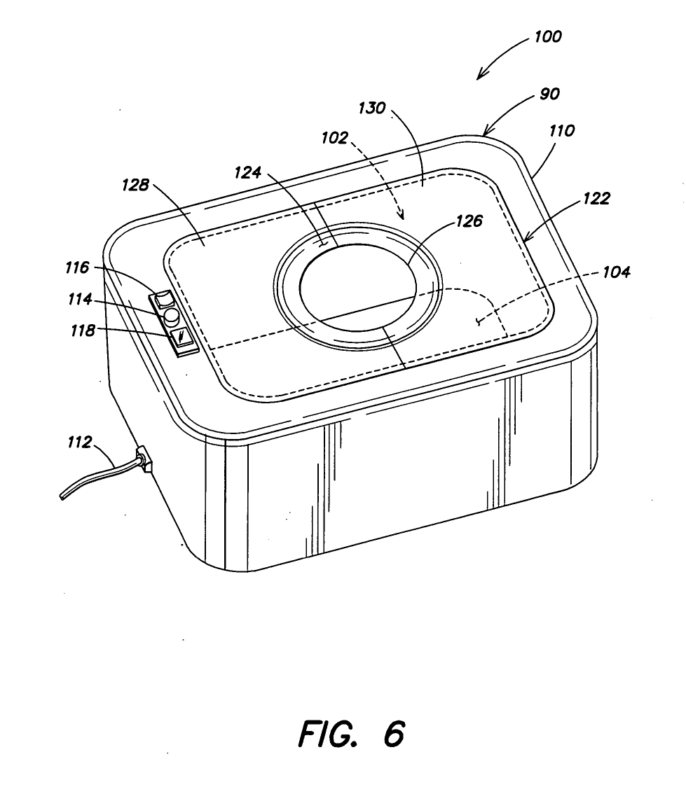

In an alternate preferred embodiment, as illustrated in a cross-sectional view in FIG. 5 and from above in FIG. 6, bath 102 is covered with an upper surface cover 122, which covers mouth 108. There is a passage 126 (which may be preferably of, but is not limited to, a circular shape) through cover 122 through which body part 120 is inserted. Cover 122 is preferably made from a durable, hard, transparent plastic, such as acrylic plastics such as Plexiglas®, and is preferably held in place by friction fitting into mouth 104. Around passage 126 on cover 122 there is preferably a lining ring 124 of a soft (preferably rubber) material so that body part is held comfortably and snugly within passage 126. For ease of insertion of the body part through passage 126, in an alternate configuration, as is shown best in FIG. 6, cover 122 may be constituted from separate portions, such as first leaf 128 and second leaf 130, as non-limiting examples, each leaf separately fitted into place sequentially around body part 120. Cover 122 encloses balls 30 and prevents the balls from “flying out” from within bath 102. In addition, cover 122 provides another surface off which balls 30 may bounce so as to move randomly within field 10 and ultimately impact on part 120 so as to impart a percussion massage.

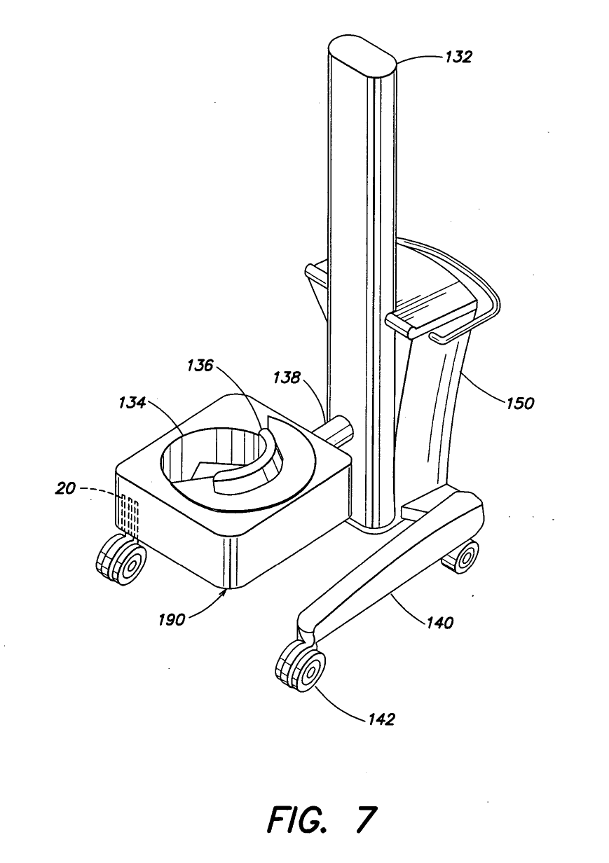

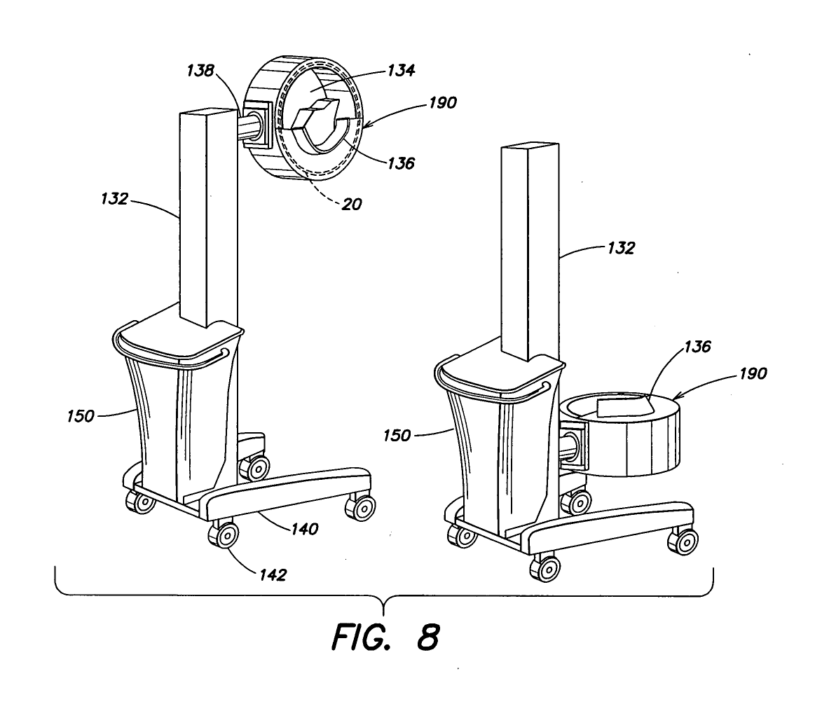

Another alternate preferred embodiment, illustrated in FIGS. 7 and 8, offers increased flexibility and ease of use for treating varying different types of body part 120. In this alternate preferred embodiment, coil 20 is not contained in the walls of a bath type housing as for the embodiments illustrated in FIGS. 3 to 6. In this preferred embodiment, magnetic coil 20 is mounted within housing 190 on a multi-position, variable height stand 132. Body part 120 to be treated is placed inside a central aperture 134 within housing 190 such that electric coil 20 surrounds body part 120. Body part 120 is thus immersed in the magnetic field but does not touch the coil. Housing 190 may be of any shape, but coil 20 within is preferably circular, as illustrated in FIG. 8. In some configurations, housing 190 has an adjustable lining rest 136, on which body part 120 may rest.

Housing 190 containing coil 20 is attached via connector 138 to stand 132 such that coil 20 hangs on a six degrees of freedom mounting. Stand 132 is attached to wheelbase 140, which has attached a plurality of wheels 142. Coil 20 can therefore be raised or lowered and rotated in any direction to accommodate different body parts such as portions of a limb, or a region of the trunk, for example, placed inside aperture 134, without changing the position of the patient to be treated. Aperture 134 varies in size from 100 mm to 1000 mm in diameter, preferably 200 mm to 600 mm, and most preferably about 300 mm to 400 mm. In different preferred embodiments, adjustment of position of coil 20 may be mechanically or electrically driven. One ordinarily skilled in the art will be capable of operatively assembling such an alternate configuration utilizing conventional components, including ventilation systems for cooling the coil and transformers as necessary, electrical motors for position adjustment, and the like. Operational controls, electronics, processors, power supply, input devices, output indicators, and the like are housed within an instrumentation body 150.

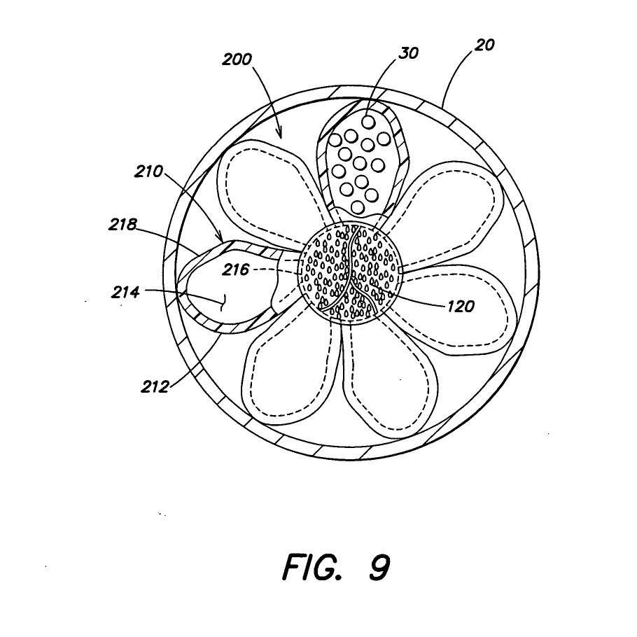

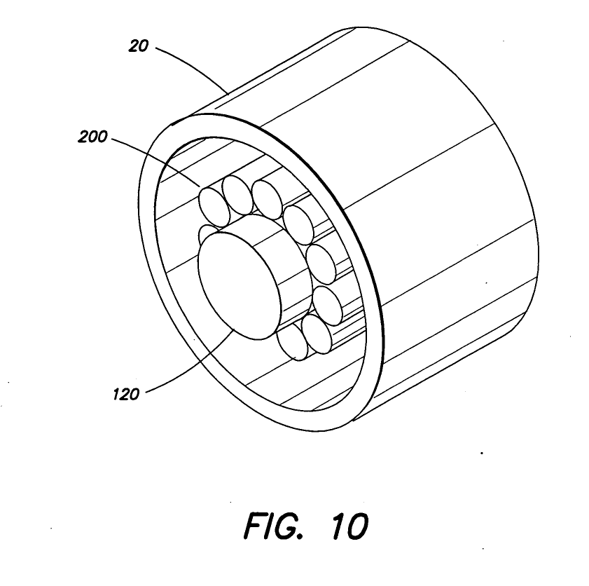

With use of the system of the preferred embodiment as illustrated in FIGS. 7 and 8, at least one, and preferably a plurality of, massage balls 30 are contained in a sleeve 200, sleeve 200 being a bag-like structure that is adapted so as to be placeable around body part 120 to be treated, as illustrated in FIGS. 9 and 10. Sleeve 200 is wrapped around body part 120 before the patient inserts it into coil 20. As described hereinabove, magnetic field 10 causes the balls to agitate freely and randomly within sleeve 200 which can fill up to the space defined between the coil and the limb, bouncing off the sides of sleeve 200.

As illustrated in FIGS. 9 and 10, in a preferred embodiment, sleeve 200 is itself composed of at least one, and preferably a plurality of separate individual chambers 210, each with an enclosure material sack 212 enclosing a pocket 214. Each enclosure material sack 212 has two portions, the portion 216 which comes in contact with body part 120 and the remainder 218 which is not in contact with body part 120. Portion 216 which is in contact with body part 120 is softer and thinner so as that the impact of the balls 30 on body part 120 is not dampened. The remaining portion 218 is thicker and more durable and rigid so as to withstand impact without wearing out and so as to allow balls 30 to more effectively rebound off this portion 218 of enclosure material sack 212 of sleeve 200 so as to better impact body part 120. Sleeve 200 is preferably transparent. Sleeve 200 is constructed from any of a number of materials including plastics, such as polyethylene, and fabrics. Each individual chamber 210 may be of various dimensions, preferably ranging from 3 to 5 cm in width by 2 to 10 cm in height by about 20 cm in length. Sleeve 200 may be of various dimensions so as to be able to enclose any body part from a portion of a limb from a wrist to a thigh, to any entire trunk or body, of a human or any animal, and may be made up of from 1 to hundreds of individual chambers, preferably about 5 to 20. Each chamber 210 contains from 1 to hundreds of balls, preferably about 1 to 20, most preferably from 5 to 10. In configurations with more than one chamber 210, the individual chambers are reversibly or irreversibly connected to one another in various manners, using at least one connection 220, including but not limited to being sewn, glued, stapled, or melted, or attached using clamps, clasps, clips, or hook and loop type closure such as VELCRO®. In alternate embodiments, sleeve 200 or at least one chamber 210 preferably has at least one vent opening 230 on enclosure material sack 212 that can be opened and fastened closed to enable the addition, removal or change of balls 30 therein.

Within rest 136 is a depression so that a portion of sleeve 200 can set within it and that segment of body part 120 covered by that portion of sleeve 200 will be impacted by free moving balls 30.

Control element 80 utilizes a processor such as a microchip by which the treatment program can be defined, controlled and executed. The program allows the operator to set the values for various parameters including but not limited to: magnetic field maximal intensity; pulse cycle times (the time in seconds that the magnetic field is on and off, repeatedly); pause time (time in seconds between pulses); treatment duration (the total time of treatment); pulse shapes (square vs. sinusoidal waves) and indirectly the speed and therefore intensity of impact of balls 30. In addition to controls for routinely turning on and off the system and starting and finishing a treatment session the control element has the capability for an emergency stop function. The individual being treated or a separate operator may activate a remote input device to pause the treatment if any question, discomfort, or inconvenience arises. Alternate configurations in which the remote input device conveys input to the system, for example, as electrical impulses traveling along a wire, as wireless transmission, as sonic transmission, as infrared transmission, as ultrasound transmission, as microwave transmission or as radio frequency transmission are within the scope of the present invention. Furthermore alternate configurations in which treatment parameter programming may be input remotely to the data processor from a remote program input device and in which treatment performance may be monitored remotely are also within the scope of the present invention. Such configurations require a uni- or bi-directional data communications channel for communication with at least one remote program input device external to instrumentation body 150, such as a personal computer. The data communication channel may be, for example, a telephone connection, a cellular telephone connection, an infrared connection, a satellite connection, cables connection, an Internet connection, a local area network connection or a radio frequency connection, or any combination thereof. Depending upon the exact nature of such configurations, these connections may require the use of additional components such as wires, antennas, receivers, transmitters, transceivers, modems, a telephony network, a cellular telephone network, an internet connection and other equipment. A portion of these additional components may already exist as part of established communication networks. One ordinarily skilled in the art will be able to assemble the required additional components using commercially available parts.

The system further features automatic controls for: monitoring safety parameters, which will stop the treatment upon any out of the ordinary occurrence, such as, an increase in temperature of the coil above a pre-defined limit, or a current intensity over a predefined maximum, as non-limiting examples. The control element will not allow the operator to program a treatment outside of a predefined set of safe parameters. The system includes at least one input device 114 and at least one display device 118 to permit entry of parameters.

FIG. 12 is a block diagram of the major features of a preferred embodiment of the circuitry of the present invention. Induction coil 20 has a thermosensor 322 connected thereto to monitor the heat generated by the coil. Thermosensor 322 is connected to overheating protection circuit 324 (to disconnect the inductor from power supply if the temperature exceeds a set maximal temperature, for example, 37° C.) which is connected to failure indicator light 326 (which is illuminated to indicate system failure) and to CPU 328. Induction coil 20 is further connected to current intensity measurement circuit 330, which includes for example a Gaussmeter sensor. Current intensity measurement circuit 330 is also connected to CPU 328. CPU 328 is connected to at least one output device, including for example, LCD 332 and indicator lights 334 and 336. In the preferred embodiment illustrated in FIG. 12, indicator light 334 indicates that a treatment is in progress, and indicator light 336 indicates that a pulse is ON. The main alternating current power input enters the circuit through fuse 338 connected to main power switch 340, and passes through first RFI filters 342 before entering insulation transformer 344. Current from insulation transformer 344 passes through overcurrent protection circuit 346, which is connected to failure indicator light 326, before reaching the start/stop relay 348. Insulation transformer 344 is also connected to power supply 350 to provide a source of 5V and 12V power. Current through start/stop relay 348 passes through second RFI filters 352 and triac 354. Triac 354 is controlled by phase control system 356, phase control system 356 itself being under the control of CPU 328. Triac 354 supplies the alternating current through current intensity measurement circuit 330 to induction coil 20. In addition to receiving input from current intensity measurement circuit 330 and overheating protection circuit 324, CPU 328 also receives input from start/stop relay 348, and input devices, such as treatment parameter input device 358. In alternate configurations, treatment parameter input device 358 may take the form of pushbuttons, a keyboard, or as described hereinabove, may be an independent device such as remote computer used by an operator such as a physician or physiotherapist to enter the treatment parameters from a site remote from the place of treatment. In alternate configurations, treatment parameter input device 358 is connected to CPU 328 by various forms of input connection 360. As described hereinabove, input connection 360 may take the form of a communications channel and may be, for example, a telephone connection, a cellular telephone connection, an infrared connection, a satellite connection, cables connection, an Internet connection, a local area network connection or a radio frequency connection, or any combination thereof Treatment session start/stop control input 362, preferably in the form of start (364) and stop (366) push buttons are connected both to start/stop relay 348 and to CPU 328. In alternate configurations, the individual being treated or a separate operator may activate a remote stop input device 370 to pause the treatment if any question, discomfort, or inconvenience arises. Alternate configurations in which the remote input device conveys input to the system, by link 368, for example, as electrical impulses traveling along a wire, as infrared transmissions, as ultrasound transmission, as microwave transmissions or as radio frequency transmissions are within the scope of the present invention.

Electromagnetic coil 20 can generate a magnetic field of up to 400 Gauss, and preferably generates a field of 200 Gauss to optimally move magnetic balls 30. The magnetic field alternates in polarity at frequencies of 1 to 1000 Hz, preferably from about 50 to 60 Hz. The pulses may be of any shape, including square waved and sinusoidal, though sinusoidal pulses are preferred. The pulses may be on continuously or intermittently, for from 1 to 60 sec, preferably from 1 to 30 sec, and most preferable from 1 to 10. The pulses may be off for from 0 to 60 sec, preferably from 0 to 30, and most preferably from 0 to 20. Total treatment time of a session may range from 1 sec to 60 minutes, preferably from 1 min to 30 minutes.

All parts of system 100 including the balls, sleeves, or housing surfaces, that come into contact with the body part 120 to be treated are made from biocompatible materials that are well known in medical applications. All circuitry is designed according to medical standards.

One should however take note that the diabetes mellitus application (in which the system of the present invention is used for management and prevention of complications of the disease and not the disease process itself) is by no means the sole potential use of the system of the present invention. Specifically envisioned as being within the scope of this invention is use of the system described hereinabove for other such applications, including but not limited to: nonunion of fractures, delayed bone healing and failed arthroses, osteoporosis, avascular necrosis of the hip, Legg-Calve-Perthes disease, degenerative spine disease, non-infectious inflammatory conditions including osteoarthritis and rheumatoid arthritis, soft-tissue injuries, various skin conditions, and pain including chronic pain conditions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A treatment system for providing electromagnetic therapy and massage comprising,
(a) an electromagnetic field inductor for producing an electromagnetic field, and
(b) at least one magnetic ball, said magnetic ball having a central magnet core covered in a substantially nonmagnetic material,
wherein said electromagnetic field is capable of causing said at least one magnetic ball to move freely and randomly within said electromagnetic field so as to massage a body part placed within said electromagnetic field.

2. The system of claim 1, wherein said electromagnetic field has a maximal induction of 200 Gauss.

3. The system of claim 1 wherein said electromagnetic field is pulsed.

4. The system of claim 1, wherein said electromagnetic field is intermittent.

5. The system of claim 1, wherein said electromagnetic field is alternating in polarity.

6. The system of claim 5, wherein said electromagnetic field alternates in polarity with a frequency of 50 to 60 Hertz.

7. The system of claim 5, wherein said electromagnetic field alternates in polarity with sinusoidal oscillations.

8. The system of claim 1, wherein said body part is of an animal.

9. The system of claim 1, wherein said body part is of a human.

10. The system of claim 1, wherein said body part is a body.

11. The system of claim 1, wherein said body part is a limb.

12. The system of claim 1, wherein said at least one magnetic ball is a plurality of magnetic balls.

13. The system of claim 1, wherein said at least one magnetic ball is spherical.

14. The system of claim 1, wherein the shape of said at least one magnetic ball is selected from the group consisting of cube, cylinder, cone, pyramid, rectangular prism, and irregular polyhedral solid.

15. The system of claim 1, wherein said at least one magnetic ball has at least one projection extending from a surface of said at least one magnetic ball.

16. The system of claim 1, wherein said substantially nonmagnetic material is soft.

17. The system of claim 1, wherein said substantially nonmagnetic material is hard.

18. The system of claim 1, wherein said substantially nonmagnetic material is selected from the group consisting of plastic, rubber, silicone epoxy, foam rubber and fabric.

19. The system of claim 1, further comprising:
(c) a housing, said housing comprising at least one wall and a base surface, said at least one wall and said base surface enclosing a bath,
wherein said electromagnetic field inductor surrounds said bath, and said at least one magnetic ball moves freely and randomly within said bath.

20. The system of claim 19, further comprising:
(d) a cover above said bath, said cover having a passage therein for insertion of said body part.

21. The system of claim 20, wherein said cover is transparent.

22. The system of claim 1, wherein said electromagnetic field inductor is circular.

23. The system of claim 1, wherein said electromagnetic field inductor is located within a housing mounted on a stand, said housing having a central aperture adapted to enable insertion of said body part.

24. The system of claim 23, where the position of said electromagnetic field inductor on said stand may be adjusted.

25. The system of claim 23, wherein said housing has a lining rest.

26. The system of claim 1, wherein said at least one magnetic ball is contained within a sleeve, said sleeve being adapted so as to be placeable around said body part.

27. The system of claim 26, wherein said sleeve has a plurality of chambers, each of said plurality of chambers having an enclosure sack with at least two portions, said first portion in contact with said body part, and a second part attached to said first part.

28. The system of claim 27, wherein said first portion of said enclosure sack is soft, and said second portion is firmer than said first portion.

29. The system of claim 26, wherein said sleeve has at least one vent for changing said at least one magnetic ball therein.

30. The system of claim 26, wherein said sleeve is transparent.

31. The system of claim 1, further comprising:
(c) a control element for automated operation of the system.

32. The system of claim 31, wherein said control element is programmable, such that at least one parameter of said operation of the system may be changed by an operator of the system.

33. The system of claim 32, wherein said at least one parameter is selected from the group consisting of maximal intensity of said electromagnetic field, pulse cycle time, pause time, and total treatment duration.

34. The system of claim 32, wherein said at least one parameter may be changed using at least one remote program input device connected to said control element by at least one communication channel.

35. The system of claim 34, wherein said at least one communication channel is selected from the group consisting of a telephone connection, a cellular telephone connection, an infrared connection, a satellite connection, cables connection, an Internet connection, a local area network connection and a radio frequency connection.

36. The system of claim 31, wherein said control element is adapted to perform an emergency stop.

37. The system of claim 36, further including a remote input device for causing said control element to perform said emergency stop.

38. The system of claim 37, wherein said remote input device conveys input to said control element by a means selected from the group consisting of electrical impulses traveling along a wire, wireless transmission, sonic transmission, infrared transmission, ultrasound transmission, microwave transmission and radio frequency transmission.

39. The system of claim 31, wherein said control element is adapted so as to prevent operation of the system if at least one safety parameter is exceeded.

40. The system of claim 39, wherein said at least one safety parameter is selected from the group consisting of inductor temperature and current intensity.

41. The system of claim 1, wherein the system is used for the treatment of vascular disease.

42. The system of claim 1, wherein the system is used for the treatment of inflammatory conditions.

43. The system of claim 1, wherein the system is used for the treatment of pain.
